SEQUENCE LISTING (18 sequences)

SEQ ID NO: 1 - DNA, 35 bp, artificial sequence
  UpDelgdhA1 - Primer to amplify upstream region of gdhA on chromosomal DNA
atatataccttagtgaccgttgagttggtgtctgc

SEQ ID NO: 2 - DNA, 41 bp, artificial sequence
  UpDelgdhA2 - Primer to amplify upstream region of gdhA on chromosomal DNA
ccttatgggatttatcttccttaagcaacgtattctttacc

SEQ ID NO: 3 - DNA, 23 bp, artificial sequence
  Upcat - Primer to amplify the P32-cat cassette on pNZ5320
taaggaagataaatcccataagg

SEQ ID NO: 4 - DNA, 23 bp, artificial sequence
  Dncat - primer to amplify the P32-cat cassette on pNZ5320
ttcacgttactaaagggaatgta

SEQ ID NO: 5 - DNA, 41 bp, artificial sequence
  DnDelgdhA1 - primer to amplify the downstream region of gdhA on chromosomal DNA
tacattccctttagtaacgtgaaatgtcagctcaaggtatt

SEQ ID NO: 6 - DNA, 35 bp, artificial sequence
  DnDelgdhA2 - primer to amplify the downstream region of gdhA on chromosomal DNA
acgtcttgagccttctttttgcctaagtctgtcag

SEQ ID NO: 7 - DNA, 20 bp, artificial sequence
  Gdh1up - primer to amplify the upstream region of gdh on chromosomal DNA
ccgttaatctgagtatcgtc

SEQ ID NO: 8 - DNA, 21 bp, artificial sequence
  moveR - primer to amplify the upstream region of gdh on chromosomal DNA
tcaatcaaagcaacacgtgct

SEQ ID NO: 9 - DNA, 28 bp, artificial sequence
  Gdh_Nco_up - primer to amplify internal fragment on pGIM008 (cmR)
catgccatggcggtggtaaagggggctc

SEQ ID NO: 10 - DNA, 27 bp, artificial sequence
  Gdh_Sac_down
ccagagctcttggcttgatcaccgctg

SEQ ID NO: 11 - DNA, 20 bp, artificial sequence
  Gdh2dw - primer to amplify the downstream region of gdh on chromosomal DNA
accatatagcaagctggcag

SEQ ID NO: 12 - DNA, 21 bp, artificial sequence
  Move1 - primer to amplify the downstream region of gdh on chromosomal DNA
tttatcgattacagctccaag

SEQ ID NO: 13 - DNA, 20 bp, artificial sequence
  glnA1up - primer to amplify the upstream region of glnA on chromosomal DNA
atcaacttggttgatcatgc

SEQ ID NO: 14 - DNA, 21 bp, artificial sequence
  FP - primer to amplify the upstream region of glnA on chromosomal DNA
cgcccgggttttcccagtcac

SEQ ID NO: 15 - DNA, 26 bp, artificial sequence
  gln_Bam_up - primer to amplify internal fragment on pJDC9 (EryR)
cgcggatccaacgaccgaattgaatg

SEQ ID NO: 16 - DNA, 25 bp, artificial sequence
  gln_Eco_down - primer to amplify internal fragment on pJDC9 (EryR)
ccggaattcaagaaccgctgcaaag

SEQ ID NO: 17 - DNA, 20 bp, artificial sequence
  glnA2dw - primer to amplify the downstream region of gdh on chromosomal DNA
catcttcatccataacatag

SEQ ID NO: 18 - DNA, 24 bp, artificial sequence
  RP_24mer - primer to amplify the downstream region of gdh on chromosomal DNA
agcggataacaatttcacacagga

(19) United States
(12) Patent Application Publication
(10) Pub. No.: US 2013/0052303 A1
(43) Pub. Date: Feb. 28, 2013

(54) Method for Improving Flavor Production in a Fermented Food Product

(71) Applicants: Willem Meindert De Vos, LM Ede (NL); Jan Sikkema, PC Zeegse (NL); Jeroen Hugenholtz, Parleiten-Geisenfeld (DE)

(72) Inventors: Margreet Ineke Pastink, PL Poortugaal (NL); Willem Meindert De Vos, LM Ede (NL); Jan Sikkema, PC Zeegse (NL); Jeroen Hugenholtz, Parleiten-Geisenfeld (DE)

(73) Assignee: PURAC BIOCHEM B.V., AC Gorinchem (NL)

(21) Appl. No.: 13/394,762

(22) PCT Filed: Sep. 8, 2010
(86) PCT No.: PCT/NL2010/050568

§ 371 Date: May 25, 2012

Related U.S. Application Data
(60) Provisional application No. 61/240,557, filed on Sep. 8, 2009.

(30) Foreign Application Priority Data
Sep. 8, 2009 (EP) 09169763.1

(51) Int. Cl. A23L 1/221; A23C 19/068; A23C 9/123; C12N 1/21; C12Q 1/32

(52) U.S. Cl. 426/38; 435/252.3; 435/26; 426/7; 426/61; 426/43

(57) ABSTRACT

The invention describes a method for improving flavor production in a fermented food product, a S. thermophilus strain wherein glutamate dehydrogenase is inactivated, as well as a food product comprising such strain. Moreover, the invention describes a method for identifying S. thermophilus strains having improved flavor production, and use thereof for improving flavor production in a fermented food product.

### FIELD OF THE INVENTION

The present invention relates to the field of microbiology and food production using microbial fermentation in which a Streptococcus thermophilus strain is used which improves flavor production in the food product.

### BACKGROUND

Streptococcus thermophilus is an important lactic acid bacterium (LAB) for the food industry. It is used for the production of Italian and Swiss cheeses, using elevated cooking temperatures and in co-cultivation with Lactobacillus delbrueckii subsp. bulgaricus for the production of yoghurt.

Furthermore, S. thermophilus is able to produce a varied amount of flavours. However, it is often used for its rapid acidification capacities. This indicates the presence of most amino acid biosynthesis and converting pathways. However, Streptococcus (S.) thermophilus LMG18311 is predicted, based on the genome, not to have a complete pentose phosphate pathway. The pentose phosphate pathway meets the need of all organisms for a source of NADPH to use in reductive biosynthesis. Most LAB possess a complete pentose phosphate pathway. Since all living organisms need NADPH, S. thermophilus needs alternative pathways to synthesize NADPH.

Based on an available genome-scale model of S. thermophilus (Pastink et al. Appl. Environm. Microbiol. 2009, vol. 75, no. 11:3627-3633), it was predicted that glutamate dehydrogenase was the most likely pathway for NADPH generation. Generally, strains exhibiting glutamate dehydrogenase (GDH) activity are capable of producing α-ketoglutarate (α-KG) from glutamate (Glu) and therefore are capable of degrading amino acids in a reaction medium containing Glu.

### SUMMARY OF THE INVENTION

The genome-scale model of S. thermophilus showed the absence of the oxidative part of the pentose phosphate pathway and the need for alternative NADPH-generating metabolic pathways. The metabolic model indicates that amino acid metabolism, and specifically glutamate dehydrogenase, would provide this NADPH.

Thus, the present inventors set out to find out which pathways are used by S. thermophilus for NADPH generation. The available genome-scale model of S. thermophilus (supra) was used and the model initially predicts glutamate dehydrogenase as most likely pathway. The present inventors constructed a knock-out of this gene and analysed the mutant obtained by growth experiments, fermentation behavior and on transcriptional level.

It was found that the disruption of GDH in S. thermophilus leads to higher activity of amino acid metabolism, demonstrated by increased ammonia-production. This was highly surprising since the product of GDH, a-ketoglutarate, is considered an essential factor in amino acid metabolism. They also found that the S. thermophilus strain having an inactive GDH has an improved flavor profile compared to the same S. thermophilus strain having an active GDH.

It is an object of the present invention to provide S. thermophilus strains with improved flavor producing capabilities. More amino acid-utilisation by S. thermophilus, would also lead to lower numbers of Lactobacillus bulgaricus in the co-cultures in yoghurt. Since L. bulgaricus is responsible for strong acidification at the end of the yogurt fermentation, this leads to less post-acidification in yogurt, and as such to a milder taste of said yogurt.

Thus, in a first aspect the present invention is concerned with a method for increasing flavor production in a fermentation broth, said method comprising the step of fermenting an fermentation medium using a S. thermophilus strain wherein glutamate dehydrogenase is inactivated.

In a further aspect, the present invention relates to a S. thermophilus strain wherein glutamate dehydrogenase is inactivated.

In yet another aspect, the invention provides for a fermentation broth comprising a S. thermophilus strain wherein glutamate dehydrogenase is inactivated.

The invention further is directed to a food product comprising a S. thermophilus strain wherein glutamate dehydrogenase is inactivated, or a fermentation broth comprising a S. thermophilus strain wherein glutamate dehydrogenase is inactivated.

The invention also pertains to a method for identifying S. thermophilus strains having increased flavor production, said method comprising the step of screening for GDH-activity in S. thermophilus strains.

In another aspect, the present invention relates to the use of a S. thermophilus strain wherein glutamate dehydrogenase is inactivated, for improving flavor production in a fermented food product, in particular yogurt or cheese.

In a final aspect, the present invention is concerned with the use of a S. thermophilus strain wherein glutamate dehydrogenase is inactivated, for obtaining reduced CFU of L. bulgaricus in yogurt compared to yogurt prepared using a S. thermophilus strain in which GDH is active.

### BRIEF DESCRIPTION OF THE DRAWINGS

The invention will be explained in more detail below, with reference to the following FIGURE:

FIG. 1 shows GC-MS analyses of the headspace of fermentation samples. S. thermophilus was grown on CDM; samples were taken at the end of the exponential growth phase (OD₆₀₀˜1.3).

### DETAILED DESCRIPTION OF THE INVENTION

The present invention relates to a method for increasing flavor production, particularly produced by S. thermophilus, in a fermentation broth, said method comprising the step of fermenting a fermentation medium using a S. thermophilus strain wherein glutamate dehydrogenase has been inactivated.

The method of the invention may comprise the steps of: a) providing a fermentation medium; b) inoculating said fermentation medium with at least a S. thermophilus strain wherein glutamate dehydrogenase has been inactivated; c) allowing fermentation to take place to obtain a fermentation broth; and optionally d) using all or part of the fermentation broth for the preparation of a food product.

The fermentation broth may serve as a flavour-providing medium itself, or flavour compounds produced may be isolated from the fermentation broth, and may subsequently be used in the preparation of a food product, feed product, and the like.

The term “increasing flavour production” as herein used refers to the production of an increased amount of at least one compound important for providing flavour to a food product. An improvement in flavour production for an S. thermophilus strain comprising an inactivated glutamate dehydrogenase (hereinafter also referred to as “GDH-inactive”) will be established in comparison to the same S. thermophilus strain in which glutamate dehydrogenase has significant glutamate dehydrogenase activity (GDH-active). As used herein, a GDH-inactive strain will at most have about 10%, such as about 8%, about 7%, about 6%, about 5%, about 4%, about 3%, preferably about 2% and even more preferably about 1%, residual GDH activity compared to the same S. thermophilus strain in which glutamate dehydrogenase has significant glutamate dehydrogenase activity (GDH-active) (‘parental strain’). The term “the same S. thermophilus strain in which glutamate dehydrogenase has significant glutamate dehydrogenase activity (GDH-active) (‘parental strain’)” is used to denote the strain from which the GDH-inactive S. thermophilus strain is derived.

Flavor production in a GDH-inactive strain will be increased, or improved, when the strain produces at least about 5%, preferably at least about 10% or about 15%, more of at least one compound important for flavor production. Compounds important for flavor production include, without limitation, acetaldehyde, methanethiol, 2-methylpropanal, 2-butanone, 3-methylbutanal, 2-methylbutanal, dimethyl disulfide (DMDS), 3-methyl-2-butenal, 2-heptanone, methional, heptanal, benzaldehyde, dimethyl trisulfide (DMTS), 2-nonanone, 2-undecanone, acetone, diacetyl, and ethylacetate. Preferably, the GDH-inactive strain produces increased amounts of at least one of acetaldehyde and 2-methylpropanal, or of both acetaldehyde and 2-methylpropanal.

The S. thermophilus strain comprising an inactivated glutamate dehydrogenase is herein also referred to as a GDH-inactive strain. The strain is preferably a recombinant strain, produced by recombinant DNA technology. Glutamate dehydrogenase may be inactivated by one or more of: deletion, insertion or mutation of the gdhA gene; replacing the gdhA promoter with a weaker promoter; antisense DNA or RNA; and siRNA. The GDH-inactive strain comprises an essentially non-functional GDH. The term “essentially non-functional GDH” as used herein means that GDH activity is negligible, and insufficient to provide sufficient NADPH to said S. thermophilus strain for growth.

The amino acid sequence of may be altered to produce an essentially non-functional GDH. To this end, amino acid residues may be deleted, inserted or mutated, to yield an inactive GDH. A mutation of the amino acid sequence is understood as an exchange of the naturally occurring amino acid at a desired position for another amino acid. Site-directed mutagenesis may be applied to, for example, alter amino acid residues in the catalytic site of GDH, amino acid residues that are important for substrate binding or cofactor binding, amino acid residues that are important for correct folding of GDH, or structurally important domains of GDH. The amino acid sequence may be mutated using site-directed mutagenesis, or may alternatively be mutated using random mutagenesis, e.g., using UV irradiation, chemical mutagenesis methods or random PCR methods. Alternatively, the gdhA gene may be partially or completely deleted or inactivated using well-known knock-out techniques. Another alternative is replacing the ghdA promoter with a weaker or inactive promoter, resulting in lack of expression of GDH. The skilled person knows how to replace the gdhA promoter with another promoter.

It is routine work for the skilled person to choose an adequate strategy to introduce a suitable modification of the gdhA gene (encoding glutamate dehydrogenase) in order not to get expression of an active GDH protein. For example, methods for in vitro mutagenesis are described in Sambrook et al. (Molecular cloning, A laboratory Manual, 2^{nd} ed., Cold Spring Harbor Laboratory Press, Cold Spring Harbor, N.Y., USA, 1989). Corresponding methods are also available commercially in the form of kits (e.g., Quikchange site-directed mutagenesis kit by Stratagene, La Jolla, USA). GDH-deletion may, for example, be accomplished by the gene replacement technology that is well known to the skilled person.

The amino acid sequences of glutamate dehydrogenase from S. thermophilus strains CNRZ1066, LMG18311, and LMD-9 are known from public databases (see, e.g., http://blast.ncbi.nlm.nih.gov/). They are identical. The GDH amino acid sequence consists of 450 amino acids. The GDH protein is highly conserved among Streptococcus species (more than 75% identity with most Streptococcus strains).

The gdhA gene may also be silenced (or “switched off”) using antisense DNA or (m)RNA or RNAi, preferably siRNA. The term gene silencing is generally used to describe the switching off of a gene by a mechanism other than genetic modification. That is, a gene which would be expressed under normal circumstances is switched off by machinery in the cell. The skilled person knows how to apply gene silencing to the present invention, and how to select and prepare a suitable gene silencing construct.

The present inventors have selected to knock out (or delete) the entire gdhA gene, as shown hereinafter in the examples section.

The fermentation medium may be any aqueous medium allowing its fermentation by S. thermophilus. “Fermentation” or “fermentation culture” refers to growth cultures used for growth of bacteria which convert carbohydrates into alcohol and/or acids, usually (but not necessarily) under anaerobic conditions. “Fermentation medium” refers to the growth medium being used for setting up the fermentation culture, while “fermentation broth” is generally used to refer to the fermented medium (i.e. during and/or after fermentation). However, both terms may be used interchangeably herein and the meaning will be clear from the context. The fermentation medium may be any fermentation medium comprising a sugar source, and a protein source. The sugar source may be any sugar that can be fermented by the S. thermophilus strain used, and includes, without limitation, lactose, sucrose, dextrose, glucose, and the like. The protein source may be any protein source, including, but not limited to, milk proteins, vegetable proteins, fish proteins, meat proteins, and the like. Particularly for the production of a fermented food product, it is preferred that the protein source is selected from milk proteins and vegetable proteins.

The fermentation broth may be any fermentation broth, but may also be a fermented food product, i.e. a liquid, semi-solid and/or solid food product (nutritional compositions), suitable for human and/or animal consumption per se.

S. thermophilus is routinely used in yogurt and cheese preparation by fermenting a milk-type base fermentation medium comprising milk proteins, e.g., milk. It is also routinely used in the preparation of soy yogurt using a soy-type base fermentation medium comprising 0.5-10% (w/w) soy protein, e.g., soy milk. S. thermophilus further requires a source of carbon and energy, such as a carbohydrate, e.g., a sugar such as lactose. Preferably, the milk-type base medium (also referred to as “milk substrate”) is natural or reconstituted milk, skimmed or otherwise, or milk-based media or media based on products of dairy origin.

This milk substrate or soy-type base medium may comprise items commonly used for the preparation of milk desserts, solid items such as fruits, chocolate chips or cereals for example, but also sweetened products or liquid chocolates.

Advantageously, the strains of the invention are employed in the preparation of all types of fermented milk and/or soy products.

Accordingly, the present invention relates to a process for the preparation of fermented dairy products in which a milk substrate is fermented with at least one S. thermophilus strain of the present invention.

Alternatively, the present invention also relates to a process for the preparation of fermented soy products in which a soy-type base fermentation medium comprising 0.5-10% (w/w) soy protein, e.g., soy milk, is fermented with at least one S. thermophilus strain of the present invention.

It is also possible to provide, in this process, for the use of a combination of the S. thermophilus strain of the invention with one or more further bacterial strains, especially with other lactic acid bacteria. “Lactic acid bacteria” (LAB) refers to bacteria, which produce lactic acid or another organic acid (such as propionic acid) as an end product of fermentation, such as, but not limited to, bacteria of the genus Lactobacillus, Streptococcus, Lactococcus, Oenococcus, Leuconostoc, Pediococcus, Carnobacterium, Propionibacterium, Enterococcus and Bifidobacterium.

Preferably, said one or more further bacterial strains are selected from Lactobacillus bulgaricus, Lactobacillus acidophilus, Lactobacillus casei and/or Bifidobacterium.

The invention also pertains to a S. thermophilus strain wherein glutamate dehydrogenase is inactivated. Said strain does not comprise a fully functional copy of glutamate dehydrogenase. It may be a recombinant strain, or a natural strain (non-gmo and non-mutant). Preferably, it is a recombinant strain. Said S. thermophilus strain is preferably food grade. “Food grade” refers to being regarded as safe for human and/or animal consumption, e.g. by the relevant regulatory authorities such as the US Food and Drug Administration (FDA). The strain may be prepared as described above. In an embodiment, the strain is S. thermophilus strain CBS 125184 that has been deposited at the Centraalbureau voor Schimmelcultures under the Regulations of the Budapest treaty (received on Sep. 3, 2009).

Moreover, the invention provides for a fermentation broth comprising a S. thermophilus strain wherein glutamate dehydrogenase is inactivated. The fermentation broth may be a fermented food product per se, such as yogurt or cheese, or the fermentation broth may be used in the preparation of a food product. “Food” or “food product” refers to liquid, semi-solid and/or solid food products (nutritional compositions), suitable for human and/or animal consumption. The food or food product may be fermented per se (“a fermented food product”), e.g., yogurt, cheese, kefir, or the like, or may comprise a fermented food product or fermentation broth prepared using the method of the present invention.

For example, the fermentation broth may be used in other food products such as liquid foods (e.g. drinks, soups, yoghurts or yoghurt based drinks, milk shakes, soft drinks, fruit drinks, fermented dairy product, meal replacers, fermented fruit and/or juice products, etc.) or solid foods/feeds (meals, meal replacers, snacks such as candy bars, animal feed, fermented dairy products, fermented food or feed products, ice products, freeze dried food additives, cheeses, etc.) or semi-solid foods (deserts, etc.). The fermentation broth may simply be added to, or used during the production process of such food products.

Alternatively, the fermentation broth may be concentrated or diluted or pre-treated prior to being used to prepare a food composition. Pre-treatments include filtration and/or centrifugation, sterilization, freeze-drying, freezing, and the like. The fermentation broth as such and/or the pre-treated fermentation broth are in essence the primary products of the above method. These primary products may be used as such, e.g., in the case of fermented food products, or may be used as a food product ingredient, i.e. a suitable amount of primary product may be used as ingredient when making a final food product. The food composition according to the invention comprises or consists of a suitable amount of primary product (fermentation broth, e.g. as such or pre-treated).

The food product or fermentation broth is preferably a fermented food product per se, including, but not limited to, a fermented dairy food product such as yogurt, cheese, kefir, buttermilk, sour cream, soy yogurt, and the like. Such food product may further comprise common ingredients for the preparation of dairy desserts, such as fruits, chocolate chips or cereals for example, but also sweetened products or liquid chocolates. The food product may further comprise common food ingredients such as emulsifiers, gelling agents, stabilizers, sweeteners, and the like. The person skilled in the art knows how to prepare a food product using the (fermented) food product of the present invention.

In an advantageous embodiment, the fermented food product is selected from yogurt or cheese. For the preparation of yogurt, a milk substrate is fermented using at least the S. thermophilus strain of the present invention and Lactobacillus delbrueckii subsp. bulgaricus. Other bacteria, such as LAB, may be added, for example to provide the yogurt probiotic properties. For the preparation of cheese, a milk substrate is fermented using at least the S. thermophilus strain of the present invention and preferably a starter culture, such as a commonly used starter cultures, and optionally adjunct cultures, for cheese manufacturing. The S. thermophilus strain of the present invention may also be part of a cheese starter culture.

The invention also provides for a method for identifying S. thermophilus strains having improved (or increased) flavor production, said method comprising the step of screening for GDH-activity in S. thermophilus strains. A further step could be the selection of one or more GDH-inactive strains, or identifying those strains that are GDH-inactive. Such strain having improved flavor production are advantageously employed in the preparation of a fermentation broth, food product or fermented food product as described above. Methods for screening for GDH-activity can be performed using methods well known in the art, for example using those method set forth in the following examples.

Also, the invention is concerned with use of a S. thermophilus strain wherein glutamate dehydrogenase is inactivated, for improving flavor production in a fermented food product, in particular yogurt or cheese. Acetaldehyde, which is overproduced in the GDH-inactive strain compared to the same GDH-active strain, gives yogurt its characteristic flavor. 2-methylpropanal adds a nutty flavor which is particularly desirable in certain types of cheese, for example, cheddar cheese.

Finally, the present invention pertains to the use of a S. thermophilus strain wherein glutamate dehydrogenase is inactivated, for obtaining reduced CFU of L. bulgaricus in yogurt compared to yogurt prepared using a S. thermophilus strain in which GDH is active. The two strains are preferably fermented under similar or identical conditions to allow a fair comparison of flavour production. The term “colony-forming unit (CFU)” is a measure of viable bacterial or fungal numbers.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition of the invention may comprise additional component(s) than the ones specifically identified, said additional component(s) not altering the unique characteristics of the invention.

The word “approximately” or “about” when used in association with a numerical value (approximately 10, about 10) preferably means that the value may be the given value of 10 plus or minus 1% of the value.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

It will be clear that the above description and FIGURE are included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

### EXAMPLES

### Materials and Methods

### Bacterial Strains, Media and Growth Conditions

The strains used in this study were Streptococcus (S.) thermophilus LMG18311, Lactococcus (L.) lactis MG1363 and Lactobacillus (Lb.) plantarum WCFS1. Cells were grown anaerobically. L. lactis and S. thermophilus were grown in M17 broth (Difco, Detroit, Mich., USA), supplemented with glucose (GM17) to a final concentration of 1% (wt/vol) at 30° C. and 42° C. respectively. S. thermophilus ΔgdhA was grown in the presence of chloramphenicol (10 μg/ml). Lb. plantarum was grown in Mann Rogosa Sharpe (MRS) broth (Merck, Whitehouse Station, N.Y., USA) supplemented with glucose to a final concentration of 1% (wt/vol) at 37° C. Escherichia coli DH5α (27) was used for one enzymatic assay and was cultivated aerobically at 37° C. on TYB medium (Difco).
For the fermentation experiment, cells were grown on chemically defined medium (CDM).
Glutamate Dehydrogenase (gdhA) Activity
Cultures of Lb. plantarum, L. lactis MG1363 (negative control) and S. thermophilus (both wild-type and gdhA knock-out) were grown until OD600˜1. Cultures were centrifuged and washed twice (4° C., 5000 rpm, 15 min) in 50 mM β-glycerophosphate (pH 7). After the second washing step, pellets were concentrated in the same buffer to OD600˜200/ml. Cell disruption by beat-beating (4×30 sec, speed 4.0, Fastprep FP120) was followed by removal of the cell debris by centrifugation (13000 rpm, 10 min, 4° C., Eppendorf Centrifuge 5417R). Because the gdhA assay is sensitive for background noise, cell free extracts were purified on a slide-a-lizer (Pierce, Rockford, Ill., USA) and were dialyzed in 50 mM β-glycerophosphate (pH 7) at 4° C. overnight. Cell free extracts were removed from the slide-a-lizer using a syringe and were immediately used for measurements of the enzyme activity.
gdhA activity was assayed with the colorimetric glutamate assay (Boehringer, Mannheim, Germany, Cat. No. 10 139 092 035). Reaction mixtures were incubated at 37° C. and contained 50 mM potassium phosphate/TEA buffer pH 9 (solution 1, kit), 1.76 U/ml diaphorase+NAD (solution 2, kit), 2 mM INT (solution 3, kit), 100 mM glutamate, 13.8 mM NADP⁺ or NAD⁺ and cell free extract. The formation of NADPH was followed spectrophotometrically by monitoring the increase of absorbance at 492 nm.

### Glucose-6-Phosphate Dehydrogenase (G6PDH) Activity

Cultures of Lb. plantarum (positive control) and S. thermophilus were grown until OD600˜1. Cultures were centrifuged and washed (5000 rpm, 15 min, 4° C.) in a Megafuge 1.0R (Heraeus Instruments, Germany) in 55 mM Tris/HCl buffer (pH 7.8). After the second washing step, pellets were concentrated in 1 ml 55 mM Tris/HCl buffer (pH 7.8) and beat-beated (4×30 sec, speed 4.0, Fastprep FP120) and centrifuged (13000 rpm, 10 min, 4° C., Eppendorf Centrifuge 5417R). G6PDH activity was assayed as described by Honjoh (Honjoh et al. 2003. Biosci. Biotechnol. Biochem. 67:1888-1896). Reaction mixtures were incubated at 25° C. and contained 55 mM Tris/HCl buffer (pH 7.8), 3.3 mM MgCl₂, 0.2 mM NADP⁺ and 3.3 mM glucose-6-phosphate and cell free extract. The formation of NADPH or NADH was followed spectrophotometrically by monitoring the increase of absorbance at 340 nm.

### Isocitrate Dehydrogenase (ICDH) Activity

Cultures of E. coli DH5α (used as a positive control) and S. thermophilus (wild-type and gdhA mutant) were grown until OD600˜1. Cells were harvested by centrifugation (5000 rpm, 15 min, 4° C.) and washed twice in 35 mM Tris/HCL buffer (pH 7.5). Cell pellet was concentrated in 1 ml 35 mM Tris/HCL buffer (pH 7.5) and beat-beated (4×30 sec, speed 4.0, Fastprep FP120). ICDH activity was assayed as described by Cvitkovitch et al (Cvitkovitch et al. 1997. J. Bacteriol. 179:650-655). Reaction mixtures were incubated at 37° C. and contained 35 mM Tris/HCl buffer (pH 7.5), 5 mM isocitrate, 3.5 mM MgCl₂ or MnCl₂, 0.35 mM NADP⁺ and cell free extract. The formation of NADPH was followed spectrophotometrically by monitoring the increase of absorbance at 340 nm.
Construction of gdhA Knock-Out for S. thermophilus
Molecular cloning techniques were carried out in accordance with standard laboratory procedures. The wild-type strain S. thermophilus LMG18311 was used for the construction of a mutant lacking a functional gdhA gene (ΔgdhA) using natural transformation of an overlapping PCR product as described by Blomqvist et al (Blomqvist et al. 2006. Appl. Environ. Microbiol. 72:6751-6756).
Construction of gdhA/glnA Knock-Out for Lb. plantarum

In Lb. plantarum, a double knock-out of both gdhA and glnA (glutamine synthetase) has been constructed following the strategy described before (Goffin et al. 2006. Appl. Environ. Microbiol. 72:7933-7940). Both genes were inactivated by single cross-over using suicide plasmids (pGIM008 and pJDC9) with insertion of an internal fragment of gene around 600 bp. The adjunction of erythromycin and chloramphenicol assure the stability of the insert. This mutant is functionally the same as the S. thermophilus gdhA mutant.

### Batch Cultivations

Fermentations were performed in duplicate in 1 L bioreactors (Applikon Biotechnology BV, The Netherlands). The fermentations were controlled by a Bio Controller ADI 1010 and by a Bio Console ADI 1025 (Applikon Biotechnology BV, The Netherlands). S. thermophilus wild-type and ΔgdhA were grown overnight in CDM and used as an inoculum of 1000 ml pH controlled CDM, the medium was 1% inoculated. The medium for the gdhA knock-out was supplemented with chloramphenicol (10 μg/ml). Strains were grown at 42° C., during growth pH was kept constant at pH=6.5, by the addition of 2.5M NaOH.
Lb. plantarum wild-type and ΔgdhA were grown overnight in CDM and used as an inoculum of 1000 ml pH controlled CDM, the medium was 1% inoculated. The medium for the gdhA knock-out was supplemented with chloramphenicol (10 μg/ml) and erythromycin (10 μg/ml). Strains were grown at 37° C., during growth pH was kept constant at pH=5.5, by the addition of 2.5M NaOH.
Cultures were stirred at a constant speed of 100 rpm. Growth was followed by measuring the cell density at 600 nm every 30 min. Samples for HPLC and RNA isolation (2×25 ml) were taken at the end of exponential phase. Samples for GC-MS analysis (3 ml) were taken at mid-exponential phase and at stationary phase.
Analysis of Genome-Wide mRNA Transcription Levels
RNA isolation
RNA was isolated, as described elsewhere (Sperandio et al. 2005. J. Bacteriol. 187:3762-3778.) with a few modifications. At mid-exponential phase, 25 ml of culture from each fermentor (duplicate samples for both restricted and complete medium) was immediately pelleted by centrifugation at 14000 rpm for 2 min at room temperature (Herolab, Unicen MR, Germany). The pellet was frozen rapidly in liquid nitrogen and stored at −80° C. until further use. The frozen pellet was resuspended in 400 μl TE and transferred to a screw cap tube containing 500 μl phenol-chloroform (5:1), 15 μl 20% sodium dodecyl sulphate, 30 μl 3M sodium acetate pH 4.8 and 0.6 g zirconium glassbeads. Cells were disrupted in a Fastprep (Savant, FP120) for 40 sec at 5.0 and the mixture was centrifuged to remove the beads (13000 rpm, 20 min, 4° C.). Subsequently, 500 μl cold chloroform was added to the supernatant followed by a centrifugation step (13000 rpm, 10 min, 4° C.). After centrifugation, 0.5 ml of the aqueous phase was used for RNA extraction using the High Pure RNA Isolation kit (Roche Diagnostics, Mannheim, Germany), following the manufacturer's protocol, except for the DNAse I treatment which we changed to 30 min at 37° C. RNA was eluted in 60 μl of elution buffer (supplied in the kit) and samples were stored at −80° C.
The concentration of RNA was checked with a ND-1000 spectrophotometer (NanoDrop Technologies, Inc., USA) and quality was checked using a 2100 Bioanalyzer (Agilent Technologies, Santa Clara, Calif., USA). Only samples with a 23S/16S ratio higher than 1.6 were used for labeling.
cDNA Synthesis and Labeling
First strand cDNA synthesis from 5 μg RNA was carried out as described previously (Saulnier et al. 2007. Appl Environ Microbiol 73:1753-1765; Serrano et al. 2007. Microb. Cell Fact. 6:29). Synthesized cDNA was purified and labeled with cyanine 3 and cyanine 5 for all samples. The quality of the labeled cDNA and its concentration were measured with the ND-1000 spectrophotometer (NanoDrop Technologies, Inc., USA).

### Hybridization

Hybridization of the labeled cDNA (0.3 μg per sample) was carried out as described previously (Saulnier et al. 2007. Appl Environ Microbiol 73:1753-1765; Serrano et al. 2007. Microb. Cell Fact. 6:29). The samples were hybridized on custom designed Agilent Technologies oligo microarrays, using the Agilent 60-mer oligo microarray processing protocol version 4.1 (Saulnier et al. 2007. Appl Environ Microbiol 73:1753-1765).

### Scanning and Data Analysis

Slides were scanned with a ScanArray Express scanner at a resolution of 5 μm. Data analysis and processing were carried out as described elsewhere (Saulnier et al. 2007. Appl Environ Microbiol 73:1753-1765; Serrano et al. 2007. Microb. Cell Fact. 6:29). Fold change (FC) is defined as 2^{M} where M=₂ log (cy5 intensity/cy3 intensity) (Serrano et al. 2007. Microb. Cell Fact. 6:29). Significantly regulated genes were defined as genes whose average p-value is less than 5% and whose M-value is equal or higher than 1.5.
Selection of scan intensity, normalization, scaling, Cyber-T and False Discovery Rate (FDR) was performed as described elsewhere (Blom et al. 2008. BMC Genomics. 9:495). In order to describe more subtle differential expressions in the microarray data that do not pass the single gene fdr criterion, a geometric mean of the FDRs of multiple genes was calculated provided that they adhered to the following criteria: (i) genes are part of the same operon, (ii) they exhibit similar differential expressions (e.g. all up-expressed in gdhA mutant), (iii) they are involved in the subsequent metabolic conversions (thus part of the same metabolic pathway).
With these criteria, we found 2 different pathways consisting of co-expressed genes that for each pathway were organized in an operon (citrate pathway and competence genes).

### Comparative Genomics

The ERGO bioinformatics suite (http://ergo.integratedgenomics.com/ERGO/) was used to compare S. thermophilus with other sequenced LAB on genome level. In particular the presence of the pentose phosphate pathway was tested and compared among the available 53 LAB genomes.

### Ammonia Measurement

The concentration of ammonia in the supernatant of fermentation samples was determined using the UV method from an Ammonia kit (R-biopharm AG, Darmstadt, Germany)

### Protein Concentration

The concentration of protein in the cell free extracts was determined using the bicinchoninic acid protein assay reagent (Pierce, Rockford, Il. USA).

### Metabolic Analysis

For the identification of volatile components in the samples, purge and trap thermal desorption cold trap gas-chromatography was used as described before (Engels. 1997. PhD thesis Wageningen University; Smit et al. 2004. Appl. Microbiol. Biotechnol. 64:396-402). The headspace samples were concentrated on a Fisons MFA815 cold trap (CE Instruments, Milan, Italy), followed by separation on a GC-8000 top gas chromatograph (CE Instruments) equipped with a CIP-SIL 5 CB low-bleed column (Chrompack, Middelburg, The Netherlands) and detection by a flame ionization detector.
Extracellular metabolites present in the supernatant of fermentation samples were measured as described elsewhere (Starrenburg and Hugenholtz. 1991. Appl. Environ. Microbiol. 57:3535-3540).

### Results

Prediction of NADPH Generation and Construction of a gdhA Knock-Out
As was described in the introduction, we used the previously developed genome-scale model of S. thermophilus (Pastink et al. 2009. Appl. Environ. Microbiol. 75:3627-3633) to search for NADPH generating pathways. S. thermophilus is predicted not to have a complete pentose phosphate pathway and cannot generate NADPH via this pathway. The model predicted that isocitrate dehydrogenase or glutamate dehydrogenase might be possible NADPH producing enzymes. The pathways where these enzymes code for, are connected via α-ketoglutarate, an important biological compound. The model predictions were tested experimentally by assaying enzyme activities. Also, the predicted absence of the PPP was verified by measuring the activity of the first enzyme of the PPP; glucose-6-phosphate dehydrogenase.
Cell free extracts of Lb. plantarum and S. thermophilus were assayed for G6PDH activity. Lb. plantarum was used as positive control, since it is known that this strain has a complete pentose phosphate pathway. The enzymatic assay indeed showed that Lb. plantarum has G6PDH activity and S. thermophilus does not have G6PDH activity (Table 1).
Cell free extracts of Lb. plantarum, L. lactis and S. thermophilus wild-type and ΔgdhA were assayed for GDH activity. L. lactis was used as negative control. Lb. plantarum and wild-type S. thermophilus possess GDH activity (Table 1). As expected, the gdhA knock-out does not have GDH activity.

TABLE 1
Activities of different enzymes tested in this study.
Note that not all organisms were included in every assay.

| | GDHA | G6PDH | ICDH |
|---|---|---|---|
| Strain | activity^{a} | activity^{a} | activity^{a} |
| S. thermophilus | 5.76 | 0 | 0 |
| LMG18311 | | | |
| S. thermophilus ΔgdhA | 0 | —^{b} | 10.2 |
| Lb. plantarum WCFS1 | 0.94 | 9.5 | 0 |
| Lb. plantarum ΔgdhA | 0 | 7.8 | 0 |
| E. coli DH5α | — | — | 371.4 |

^{a}Enzyme activity expressed as 1 nmol NADPH (min · mg protein)⁻¹, average of two duplicates.
—^{b}, not done.

Cell free extracts of S. thermophilus wild-type and ΔgdhA and E. coli DH5α were assayed for isocitrate dehydrogenase activity. E. coli was used as positive control and indeed has ICDH activity. The ICDH of E. coli is a key regulatory enzyme in the TCA cycle and therefore a high activity of this enzyme is observed. ICDH activity is not observed for the wild-type of S. thermophilus), whereas the gdhA mutant has ICDH activity (Table 1).
Based on this knowledge and on the observation that the wild-type of S. thermophilus can utilize glutamate (Tanous et al. 2002. Antonie Van Leeuwenhoek. 82:271-278), a glutamate dehydrogenase mutant was constructed, using natural transformation of an overlapping PCR product as described by Blomqvist et al. (supra). The gdhA open reading frame was swapped by the P32-cat cassette conferring resistance to chloramphenicol. The primers used in this study are listed in Table 2 and all PCR reactions were performed with the Fhusion polymerase (New England Biolabs Inc, Ipswich, Mass., USA).

TABLE 2
Primers used in this study for construction of the gdhA
knock-out in S. thermophilus

| Primer | Name | Sequence^{a} |
|---|---|---|
| Primers to amplify the upstream | UpDelgdhA1 | ATATATACCTTAGTGACCGTTGAGTTGGTGTCTG |
| region of gdhA on chromosomal | | C |
| DNA | | |
| | UpDelgdhA2 | CCTTATGGGATTTATCTTCCTTAA |
| | | GCAACGTATTCTTTACC |
| Primers to amplify theP32-cat | Upcat | TAAGGAAGATAAATCCCATAAGG |
| cassette on pNZ5320 | | |
| | Dncat | TTCACGTTACTAAAGGGAATGT |
| | | A |
| Primers to amplify the | DnDelgdhA1 | TACATTCCCTTTAGTAACGTGAAATGTCAGCT |
| downstream region of gdhA on | | CAAGGTATT |
| chromosomal DNA | | |
| | DnDelgdhA2 | ACGTCTTGAGCCTTCTTTTTGCCT |
| | | AAGTCTGTCAG |

^{a}Bold and underlined sequences are complementary

The upstream and downstream recombination fragments (1.5 kb) of gdhA were amplified by PCR using UpDelgdhA1/UpDelgdhA2 and DnDelgdhA1/DnDelgdhA1 primer pairs, respectively. The P32-cat cassette from pNZ5320 (a derivative of pNZ5318, removal of 1p291 fragment) (Lambert et al. 2007. Appl. Environ. Microbiol. 73:1126-1135) was amplified by PCR using Upcat and Dncat primers. The 3 overlapping PCR products were mixed in equimolar concentration, joined together by PCR using primers UpDelgdhA1/DnDelgdhA2, and the PCR mix was then used for natural transformation. The mutant genotype was confirmed by PCR with primers located upstream and downstream of the recombination regions. As a control, we used a pentose phosphate pathway positive LAB, Lb. plantarum, for which a similar gdhA mutation was constructed. The primers used for the construction of this mutant are listed in Table 3.

TABLE 3
Primers used in this study for construction of the gdhA/glnA
knock-out in Lb. plantarum

| Primer | Name | Sequence |
|---|---|---|
| Primers to amplify the upstream | Gdh1up | CCGTTAATCTGAGTATCGTC |
| region of gdh on chromosomal DNA | | |
| | moveR | TCAATCAAAGCAACAC |
| | | GTGCT |
| Primers to amplify internal | Gdh_Nco_ | CATGCCATGGCGGTGGTAAAGGGGG |
| fragment on pGIM008 (cmR) | up | CTC |
| | Gdh_Sac_ | CCAGAGCTCTTGGCTT |
| | down | GATCACCGCTG |
| Primers to amplify the downstream | Gdh2dw | ACCATATAGCAAGCTGGCAG |
| region of gdh on chromosomal DNA | | |
| | Move1 | TTTATCGATTACAGCTC |
| | | CAAG |
| Primers to amplify the upstream | glnA1up | ATCAACTTGGTTGATCATGC |
| region of glnA on chromosomal DNA | | |
| | FP | CGCCCGGGTTTTCCCA |
| | | GTCAC |
| Primers to amplify internal | gln_Bam_ | CGCGGATCCAACGACCGAATTGAAT |
| fragment on pJDC9 (EryR) | up | G |
| | gln_Eco_ | CCGGAATTCAAGAACC |
| | down | GCTGCAAAG |
| Primers to amplify the downstream | glnA2dw | CATCTTCATCCATAACATAG |
| regionof gdh on chromosomal DNA | | |
| | RP_24mer | AGCGGATAACAATTTC |
| | | ACACAGGA |

### Metabolic Response

Fermentation samples were analysed on HPLC (Table 4). The wild-type shows homolactic growth, as was observed and described before (Hols et al. 2005.FEMS Microbiol. Rev. 29:435-463). The gdhA mutant also mainly produces lactate and small amounts of formate, and it consumes citrate. Less glucose is consumed by the mutant than by the wild-type, at the same growth rate, this may indicate a more efficient growth.

TABLE 4
HPLC analyses of fermentation cell supernatants. S. thermophilus and Lb. plantarum were
grown on CDM; samples were taken at the end of the exponential growth phase

| Metabolite (mM)^{a} | | Citrate | Lactate | Formate | Acetate | Ethanol | Glucose |
|---|---|---|---|---|---|---|---|
| S. thermophilus | CDM (blanc) | 2.08 | ND | ND | 11.07 | ND | 24.31 |
| | Wild-type | 2.13 | 27.62 | 0.66 | 12.26 | ND | 8.74 |
| | gdhA knock-out | 1.19 | 23.44 | 1.50 | 12.73 | ND | 12.61 |
| Lb. plantarum | CDM (blanc) | 2.20 | ND | 0.65 | 9.61 | ND | 28.33 |
| | Wild-type | 0.82 | 16.21 | 0.00 | 4.25 | ND | 0.00 |
| | gdhA knock-out | 0.91 | 17.50 | 0.00 | 4.51 | ND | 0.12 |

^{a}Average of 2 duplicates,
N.D. not detected.

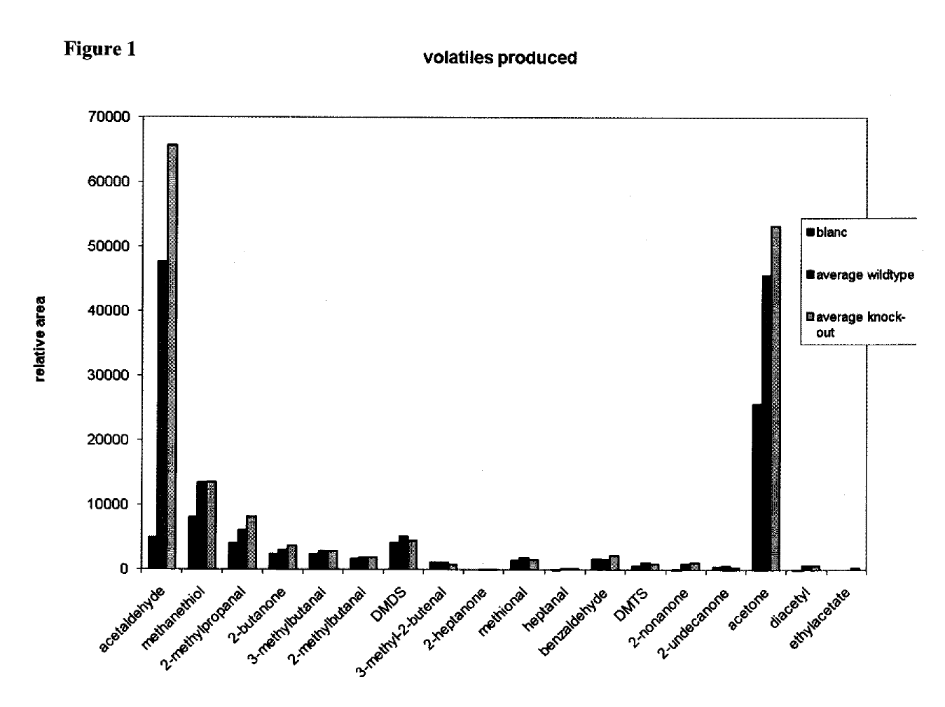

The volatile profiles are similar for the two strains (FIG. 1); however the gdhA mutant produces more acetaldehyde, acetone and 2-methylpropanal than the wild-type.
The gdhA also consumes more threonine, and this can point to acetaldehyde production from threonine conversion by threonine aldolase. Also, the gdhA mutant produces more propanone than the wild-type does, propanone can be formed as part of glycolysis. Some aldehydes such as 2-methylpropanal and 3-methylbutanal are found in increased concentrations in samples from the gdhA mutant. These aldehydes are produced during valine and leucine metabolism respectively and HPLC data indeed show increased consumption of the branched chain amino acids by the mutant compared to the wild-type. HPLC analyses of amino acids in the same samples (Table 5) shows that all amino acids are more consumed by the gdhA mutant than by the wild-type.

TABLE 5
HPLC analyses (amino acids) of fermentation cell supernatants. S. thermophilus was
grown on CDM; samples were taken at the end of the exponential growth phase
(same biomass).

| | Amino acid uptake (medium-sample) μM^{a} | | | |
|---|---|---|---|---|
| | S. thermophilus | gdhA | Lb. plantarum | gdhA |
| | Wild-type | knock-out | Wild-type | knock-out |
| Alanine | 510.5 | 908.5 | 838.6 | 930.3 |
| Arginine | 112.5 | 194 | N.D. | N.D. |
| Asparagine | −262.5 | 946 | −191.7. | −115.1 |
| Aspartic acid | 93.5 | 280.5 | −2773.3 | 266.4 |
| Cysteine | 1059 | 1135.5 | N.D. | N.D. |
| Glutamine | 481.5 | 838 | 2561.1 | 4309.5 |
| Glutamic acid | 276.5 | 744.5 | 1613.2 | 1042.2 |
| Glycine | 188 | 470 | 970.5 | 1009.1 |
| Histidine | 63 | 220 | 445.9 | 463.4 |
| Isoleucine | 299.5 | 551 | 829.0 | 818.2 |
| Leucine | 475 | 1015 | 1815.5 | 1655.2 |
| Lysine | 219.5 | 520 | 1408.3 | 1650.5 |
| Methionine | 173.5 | 230 | 444.0 | 447.3 |
| Phenylalanine | 266.5 | 506.5 | 1027.5 | 1026.0 |
| Proline | 464.5 | 1190.5 | 1924.7 | 1980.5 |
| Serine | 356 | 891.5 | 2150.6 | 2340.5 |
| Threonine | 153 | 517.5 | 969.6 | 1006 |
| Tryptophan | 215.5 | 495.5 | 33.4 | 83.0 |
| Tyrosine | 758.5 | 826 | 994.6 | 1062.7 |
| Valine | 347.5 | 731 | 1336 | 1349.3 |

^{a}Average of 2 duplicates

In addition, the mutant shows an increased production (almost 3×) of ammonia compared to the wild-type (Table 6) and this fits well with the increased amino acid consumption. This probably indicates amino acid degradation.

TABLE 6
Measured ammonia concentrations in fermentation cell
supernatants. S. thermophilus was grown on CDM;
samples were taken at the end of the exponential
growth phase (same biomass).

| | NH₃ production |
|---|---|
| | (mM) |
| Wild-type | 2.2 |
| gdhA knock-out | 6.0 |

In the case of Lb. plantarum, fermentation samples were analyzed following the same procedure as was used for S. thermophilus (Table 4). The gdh/glnA mutant and the wild-type do not show a difference in the primary metabolism; lactate, formate and acetate are produced in similar amounts. Furthermore, amino acid measurements show a similar utilisation by the mutant of the different amino acids with an exception for aspartate (Table 5). The volatile profiles of the wild-type and the gdhA/glnA mutant were nearly identical (data not shown).

### Transcriptome Analysis

The impact of the absence of the ghdA gene on the gene expression was studied with microarrays. For differentially regulation, we only selected those genes that satisfied the following criteria (i) ratio≧1.25 and (ii) FDR-value<0.05. With these criteria, we found 142 genes to be differentially expressed (Table S1). Logically, glutamate dehydrogenase is heavily down regulated in the gdhA mutant. The ΔgdhA requires alternative pathways to produce NADPH; a prediction from the recently developed Simpheny model (Pastink et al. 2009. Appl. Environ. Microbiol. 75:3627-3633) was isocitrate dehydrogenase. The array data showed an up regulation of the citrate metabolism (geometric mean FDR<0.05; average ratio≧1.25); methylcitrate synthase, aconitate synthase and isocitrate dehydrogenase. This up-regulation also corresponds with the consumption of citrate (HPLC analysis), and the increased ICDH activity and may indicate the importance of isocitrate dehydrogenase for NADPH. Furthermore, some parts of the amino acid metabolism are affected in the gdhA mutant; some amino acid transporters are up-regulated in the mutant and a branched chain amino acid exporter is down-regulated. Histidine ammonia lyase is down-regulated, this enzyme is part of the nitrogen metabolism. Phosphoserine aminotransferase is highly up-regulated in the gdhA mutant; this enzyme catalyzes the formation of glutamate and phosphonooxypyruvate from O-phospho-L-serine and 2-oxoglutarate.

Co-expressed genes (geometric mean FDR<0.05; average ratio≧1.25) involved in competence were down-regulated in the mutant compared to the wild-type.

### Comparative Genomics

The ERGO bioinformatics suite was used to compare the available sequenced LAB on the absence of the pentose phosphate pathway. S. thermophilus is not the only LAB with an incomplete PPP. Actually, almost all Streptococci with the exception of a few S. suis and S. pneumonia strains, lack the oxidative part of the 132 gdhA mutation in S. thermophilus PPP. All streptococci share the same common ancestor, but this ancestor splits in different branches. The event of the gene loss of the PPP genes in most streptococci probably occurred parallel and for a functional reason. S. thermophilus is known for its fast growth, and the event of gene loss among different streptococci does not seem unique and does not result in growth delay.

1. A method for increasing flavor production in a fermentation broth, said method comprising the step of fermenting a fermentation medium using a S. thermophilus strain wherein glutamate dehydrogenase is inactivated and allowing fermentation to take place to obtain a fermentation broth.

2. A method according to claim 1, wherein the glutamate dehydrogenase is inactivated by one or more of:
deletion, insertion or mutation of the gdhA gene;
replacing the gdhA promoter with a weaker promoter;
antisense DNA or RNA; and
siRNA.

3. A method according to claim 1, wherein the fermentation broth is a food product.

4. A method according to claim 1, wherein the fermentation broth is a fermented food product.

5. A S. thermophilus strain wherein glutamate dehydrogenase is inactivated.

6. The S. thermophilus strain according to claim 5, wherein the S. thermophilus strain is food grade.

7. The S. thermophilus strain according to claim 6 wherein the strain is CBS 125184.

8. A fermentation broth comprising the S. thermophilus strain according to claim 5.

9. A fermentation broth according to claim 8, wherein the fermentation broth is a fermented food product.

10. A fermentation broth according to claim 9, wherein the fermented food product is yogurt or cheese.

11. A food product comprising the S. thermophilus strain according to claim 5.

12. A method for identifying a S. thermophilus strain having improved flavor production, said method comprising the step of screening for decreased GDH-activity in the S. thermophilus strain.

13. A method of improving flavor production in a fermented food product comprising the step of using the S. thermophilus strain according to claim 5 in the fermented food product.

14. A method of preparing yogurt comprising the step of preparing yogurt using the S. thermophilus strain according to claim 5, wherein the colony-forming units of L. bulgaricus in the yogurt prepared using the S. thermophilus strain are reduced compared to the colony-forming units of L. bulgaricus in yogurt prepared using a S. thermophilus strain in which GDH is active.

15. The S. thermophilus strain according to claim 5, wherein the glutamate dehydrogenase is inactivated by one or more of:
deletion, insertion or mutation of the gdhA gene;
replacing the gdhA promoter with a weaker promoter;
antisense DNA or RNA; and
siRNA

16. The method of claim 13, wherein the fermented food product is yogurt or cheese.

17. A food product comprising the fermentation broth according to claim 8.
